(19) United States
(12) Patent Application Publication
(10) Pub. No.: US 2026/0192128 A1
(43) Pub. Date: Jul. 9, 2026

(54) ELECTRONIC DEVICE AND METHOD FOR INDICATING A TREATMENT SITE THEREOF

(71) Applicant: REJUVENOR INC., Chuncheon-si (KR)

(72) Inventor: Bong Kyun AHN, Seoul (KR)

(21) Appl. No.: 19/555,190

(22) Filed: Mar. 3, 2026

Related U.S. Application Data
(63) Continuation of application No. PCT/KR2024/012996, filed on Aug. 30, 2024.

(30) Foreign Application Priority Data
Sep. 4, 2023 (KR) 10-2023-0116942

(51) Int. Cl. A61N 5/06; A61N 5/067; A61B 17/00

(52) U.S. Cl. CPC A61N 5/0616; A61N 5/067; A61B 2017/00057; A61B 2017/00128; A61B 2017/00199; A61N 2005/0626

(57) ABSTRACT

An electronic device according to an embodiment includes a display, a camera module, and a processor functionally connected to the display and the camera module. While an energy irradiator irradiates a first energy to a treatment site of a subject for a designated time, the processor acquires information on the treatment site using the camera module oriented toward the treatment site, identifies a treatment region corresponding to an irradiation region of the first energy based on a feature change of the treatment site according to the acquired information, and displays, on the display, a processed image corresponding to the treatment site of the subject such that the treatment region is distinguished from other regions.

### CROSS-REFERENCE TO RELATED APPLICATION

This application is a continuation of PCT Patent Application No. PCT/KR2024/012996 filed on August 30, 2024, which claims priority to Korean Patent Application No. 10-2023-0116942, filed on September 4, 2023, in the Korean Intellectual Property Office, the disclosure of which is incorporated by reference herein in its entirety.

### BACKGROUND

### [TECHNICAL FIELD]

Various embodiments disclosed herein relate to medical treatment information guidance technology.

### [BACKGROUND ART]

Lasers (LASER: Light Amplified by Stimulated Emission of Radiation) and various types of energy delivery devices are widely used in the medical field. Lasers and various types of energy delivery devices are used for various treatments based on a principle in which energy is absorbed by human tissue and converted into thermal energy, thereby causing thermal damage and chemical reactions in cells or tissues.

Initially, lasers having high output energy with tissue-destructive effects were used for surgical treatments such as tissue incision, coagulation, and hemostasis. Recently, lasers have been used in dermatological cosmetic procedures for obesity improvement, scar removal, or treatment of pigmentation (spots, erythema, melasma, and freckles). Various types of energy delivery devices are also used for skin texture, elasticity, and lifting.

### SUMMARY

### [Problem to be Solved]

A practitioner (medical staff) irradiates a treatment site by manually adjusting a spot size, a wavelength, an irradiation time, an irradiation method, a delivery frequency per second (Hz), and an energy intensity according to a treatment target and a treatment purpose using a laser irradiator and various types of energy delivery devices (e.g., radio frequency or ultrasound). However, dermatological cosmetic lasers and various types of energy delivery devices (other examples: radio frequency or ultrasound) may not produce noticeable changes on the skin (such as redness, scab formation, or marks) immediately after being irradiated onto the skin or after energy is delivered. In addition, in the case of lasers having certain wavelengths, the wavelength itself belongs to an infrared or ultraviolet region that is invisible to the naked eye, making it difficult to even identify a currently treated site. To address this, commercially available laser equipment employs a method of additionally irradiating a point beam (laser pointer) together with a treatment laser to at least help identify where the laser is currently being irradiated. Nevertheless, it is difficult for the practitioner to determine how much laser energy has been irradiated to which treatment site, and which areas have been irradiated with the laser and which areas have not.

Various embodiments disclosed herein can provide an electronic device capable of displaying a laser and various types of energy delivery device treatment sites and delivered energy amounts on a display, and a method for displaying a treatment site and a delivered energy amount thereof.

### [Means for Solving the Problem]

An electronic device according to an embodiment disclosed herein includes a display, a camera module, and a processor functionally connected to the display and the camera module. The processor is configured to, while an energy irradiator irradiates a first energy to a treatment site of a subject for a designated time, cause the camera module to face the treatment site to acquire information on the treatment site using the camera module, identify a treatment region corresponding to an irradiation site of the first energy based on a feature change of the treatment site according to the acquired information, and display, on the display, a processed image corresponding to the treatment site of the subject such that the treatment region is distinguished from other regions.

### [Effects of the Invention]

According to various embodiments disclosed herein, a laser and various types of energy delivery device treatment sites and delivered energy amounts can be visually displayed. In addition, various effects that are directly or indirectly identified through this document can be provided.

### [BRIEF DESCRIPTION OF THE DRAWINGS]

FIGS. 1A, 1B, and 1C are reference diagrams illustrating a light treatment principle according to an embodiment.

FIG. 2 is a diagram illustrating an example of use of an electronic device according to an embodiment.

FIG. 3 is a detailed block diagram of an electronic device according to an embodiment.

FIG. 4 is a diagram illustrating an example of displaying an irradiation region of a first energy according to an embodiment.

FIG. 5 is a flowchart illustrating a method for displaying a treatment site according to an embodiment.

In relation to the description of the drawings, the same or similar reference numerals may be used for the same or similar components.

### [DETAILED DESCRIPTION]

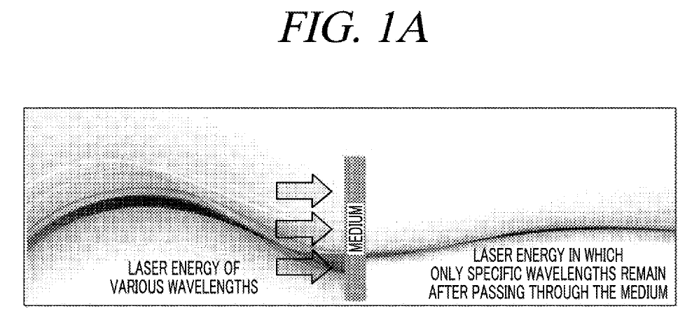

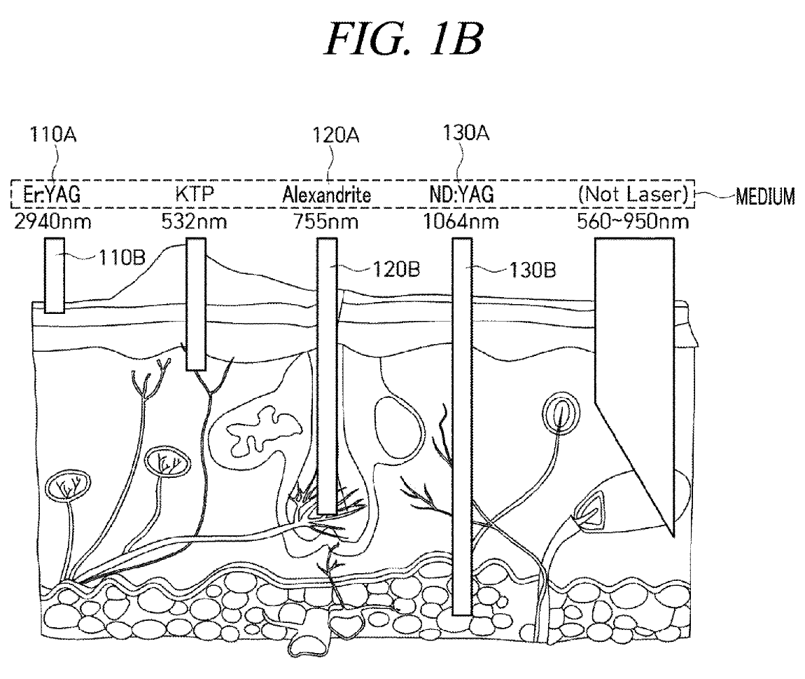

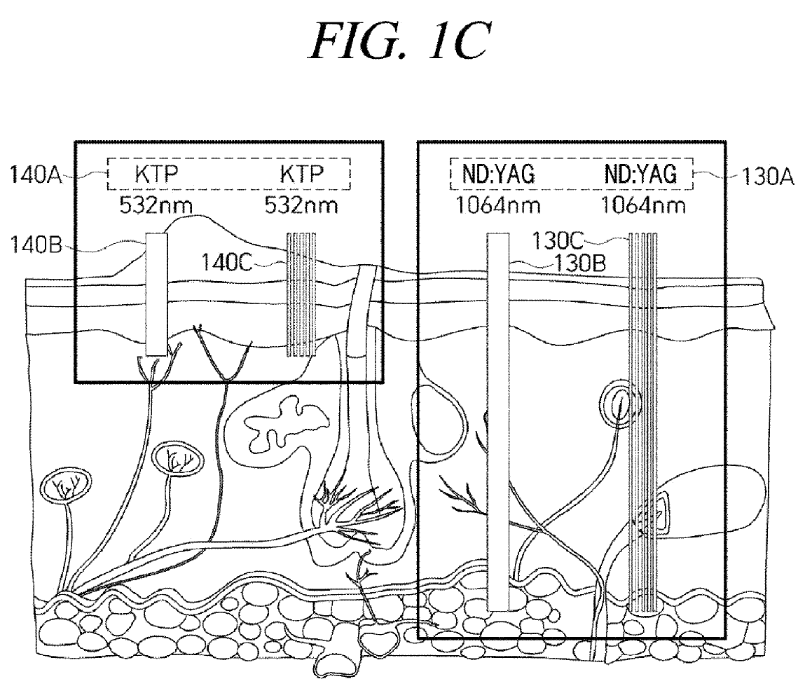

FIGS. 1A, 1B, and 1C are reference diagrams related to a light treatment principle according to an embodiment.

In light-based therapeutic procedures, light energy must be delivered to a depth at which a lesion of a subject is located in order to treat the lesion. A depth to which light (or wavelength) energy penetrates into the skin (a travel distance of light energy) may be determined according to a wavelength thereof. Therapeutic light (or wavelength) may use ultrasound, radio frequency, or a laser; however, for convenience of description, the present document mainly describes a case in which the therapeutic light (or energy) is a laser.

Referring to FIG. 1A, laser energy of various wavelengths (energy intrinsic to a laser) may be converted into laser energy of a specific wavelength as the laser energy passes through a specific medium that allows only a specific wavelength to pass therethrough. When laser energy of a specific wavelength is irradiated onto skin, the laser energy may travel to a depth within the skin corresponding to the wavelength and deliver the intrinsic laser energy at the depth. Such a medium may be regarded as a core component of a laser device to the extent that it replaces a name of the laser. The medium may include, for example, erbium-doped yttrium aluminum garnet (Er:YAG), alexandrite, or neodymium-doped yttrium aluminum garnet (ND:YAG).

Referring to FIG. 1B, skin may include an epidermis, a dermis, and subcutaneous tissue. Pigmented lesions such as melasma mainly occur in the epidermal layer, wrinkle and scar lesions mainly occur in the dermal layer, and lesions that mainly cause changes in contour occur in the subcutaneous tissue.

Referring to FIGS. 1A and 1B, an erbium-doped yttrium aluminum garnet (Er:YAG) medium (110A) allows only a 2940 nm wavelength of a laser to pass therethrough, thereby delivering laser energy to a surface of the skin (the epidermis). Accordingly, an Er:YAG laser (110B) may be mainly used to treat lesions occurring in the epidermis. For example, an alexandrite medium (120A) allows only a 755 nm wavelength of a laser to pass therethrough, thereby delivering laser energy to a hair follicle depth (the dermis) within the skin. Accordingly, an alexandrite laser (120B) may be mainly used to treat lesions occurring in the dermis. For example, a neodymium-doped yttrium aluminum garnet (ND:YAG) medium (130A) allows only a 1064 nm wavelength of a laser to pass therethrough, thereby delivering laser energy to deep subcutaneous tissue within the skin. Accordingly, an ND:YAG laser (130B) may be mainly used to treat lesions occurring in the subcutaneous tissue.

Referring to FIG. 1C, even lasers having the same wavelength may exhibit different effects depending on an irradiation method (pulse irradiation or continuous irradiation). For example, a laser irradiated strongly and for a long duration in a long-pulse method may heat a lesion area, thereby burning and removing the lesion by heat. Accordingly, a laser irradiated in the long-pulse method has an effect of treating vascular diseases such as redness and facial flushing. On the other hand, for example, a laser irradiated weakly and for a short duration in a Q-switch method may break a lesion by a shock wave caused by vibration. Accordingly, a laser irradiated in the Q-switch method has an effect of treating pigmented diseases such as hyperpigmentation, freckles, and pigmented melasma.

A 532 nm wavelength laser (140B, 140C) that has passed through a KTP medium (140A) may be irradiated in a long-pulse method or a Q-switch method. A 532 nm wavelength laser (140B) irradiated in the long-pulse method may treat vascular diseases occurring in a dermal layer, and a 532 nm wavelength laser (140C) irradiated in the Q-switch method may treat pigmented diseases in the dermal layer. In the Q-switch method, a pulse time interval (hereinafter, also referred to as an irradiation time) may be adjusted, and when the pulse time interval is short, an energy density increases, and when the pulse time interval becomes longer, the energy density may decrease.

Similarly, a 1064 nm wavelength laser (130B, 130C) that has passed through an ND:YAG medium (130A) may treat vascular diseases and pigmented diseases, respectively, in a subcutaneous tissue layer.

As described above, a laser has a specific wavelength after passing through a medium, and intrinsic energy of the laser is delivered to a depth within skin corresponding to the wavelength, thereby treating different lesions according to an irradiation method.

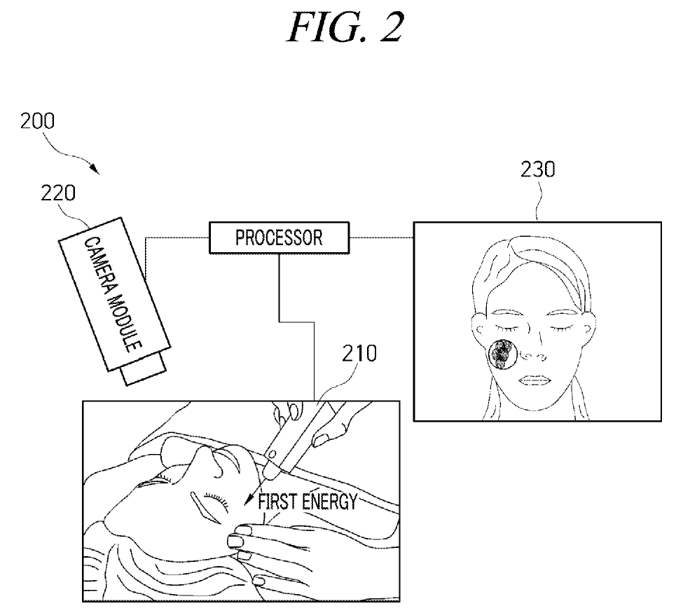

FIG. 2 illustrates an example of use of an electronic device according to an embodiment.

Referring to FIG. 2, an electronic device (200) according to an embodiment may include a camera module (220) and a display (230).

In an embodiment, the electronic device (200) may include a laser irradiator (210) and may be laser equipment configured to irradiate a laser having a designated wavelength. Alternatively, the electronic device (200) may be a component separate from an energy irradiator (210). In the present document, a case in which the electronic device (200) includes the energy irradiator (210) is mainly described as an example. However, the present disclosure is not limited thereto.

According to an embodiment, the energy irradiator (210) may irradiate, continuously or in pulses, laser energy having a designated wavelength that has passed through a designated medium (hereinafter, also referred to as a “first energy”). The first energy may include various types of energy such as laser, ultrasound, radio frequency, intense pulsed light (IPL), or plasma. In the present document, for convenience of description, a case in which the energy irradiator (210) irradiates laser energy is mainly described as an example. However, the present disclosure is not limited thereto. The laser energy having the designated wavelength may provide a treatment effect corresponding to a wavelength, an irradiation time, an irradiation method, and an energy intensity with respect to a treatment site of a subject. The treatment site may vary according to a lesion location, but in the present document, a case in which the treatment site is a face is described as an example.

According to an embodiment, while irradiating the first energy, the electronic device (200) may irradiate a point beam to at least a portion of a same region as the first energy. The point beam may be a visible light laser (hereinafter, also referred to as a “point beam”).

According to an embodiment, while irradiating the first energy and the point beam, the electronic device (200) may cause the camera module (220) to face a treatment site of a subject and acquire information on the treatment site using the camera module (220). The electronic device (200) may identify a treatment region of the subject by identifying features (e.g., color, shape, pattern, movement, or other changes) of a skin contact region of the point beam or the energy irradiator (210) based on the information acquired through the camera module (220). The electronic device (200) may display, on the display (230), the identified treatment region by distinguishing the treatment region by color and density in a processed image including an entire treatment site of the subject (or at least a currently treated region).

According to an embodiment, a laser irradiated by the electronic device (200) (other examples: radio frequency or ultrasound) may not produce noticeable changes (such as redness, scab formation, or marks) on skin immediately after being irradiated onto skin of a subject. Accordingly, a user (practitioner) of a conventional laser device has a limitation in that the user must rely only on memory to determine how much laser energy has been irradiated to which region of the subject. However, the electronic device (200) according to an embodiment may identify a laser irradiation region of the subject based on features of a point beam (laser pointer) irradiated together with a treatment laser (the first energy) or a skin contact region of the energy irradiator (210), and display the laser irradiation region on the display (230), thereby supporting the practitioner to accurately identify the treatment site.

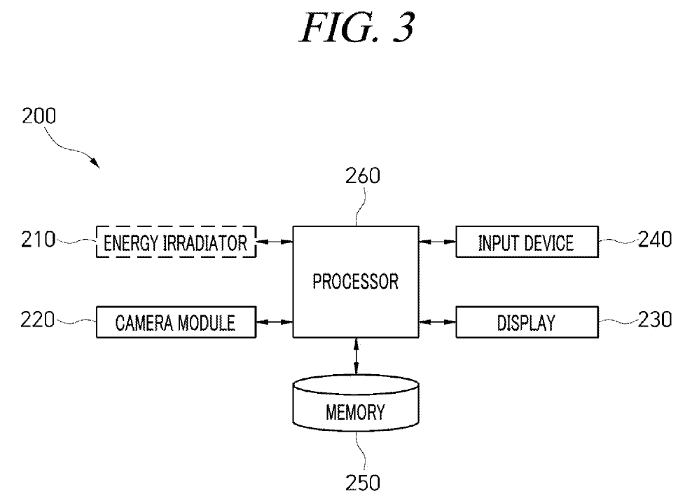

FIG. 3 is a block diagram illustrating a configuration of an electronic device according to an embodiment.

Referring to FIG. 3, an electronic device (200) according to an embodiment may include an energy irradiator (210), a camera module (220), a display (230), an input device (240), a memory (250), and a processor (260). In an embodiment, the electronic device (200) may omit some components or may further include additional components. For example, the energy irradiator (210) may be omitted. In addition, some of the components of the electronic device (200) may be combined into a single entity while performing the same functions as those of the components before combination. For example, some of the components (230, 240, 250, 260) of the electronic device (200) excluding the camera module (220) and the energy irradiator (210) may be provided in a first device, and the remaining components (230, 240, 250, 260) of the electronic device (200) excluding the energy irradiator (210) and the camera module (220) may be provided in a second device. In this case, the first device and the second device may transmit and receive data to and from each other through a designated communication channel.

According to an embodiment, the energy irradiator (210) is provided to irradiate a first energy. The first energy may provide a designated therapeutic effect to irradiated skin. The first energy may include laser energy. For example, the energy irradiator (210) may generate the first energy by allowing intrinsic laser energy to pass through a designated medium. The designated medium may include at least one of all types of laser generation media usable for skin treatment, such as CO₂, erbium-doped yttrium aluminum garnet (Er:YAG), alexandrite, ruby, diode, or neodymium-doped yttrium aluminum garnet (ND:YAG).

According to an embodiment, the energy irradiator (210) may be further provided to irradiate a point beam. For example, when the first energy is laser energy, the energy irradiator (210) may further irradiate a point beam. The point beam may be a visible-light laser point beam having a designated color (e.g., green or red). The point beam may be irradiated to at least a portion of the same region as the first energy for a designated time (e.g., the same time as the first energy). For example, the point beam may be irradiated to a region overlapping an entire region to which the first energy is irradiated. As another example, the point beam may be irradiated to overlap at least a portion (e.g., a center) of a region to which the first energy is irradiated with a designated shape and a designated size. However, the present disclosure is not limited thereto. When ultrasound or radio frequency energy is irradiated instead of the first energy, the energy irradiator (210) may not include a component or member for irradiating the point beam.

According to an embodiment, under control of the processor (260), the energy irradiator (210) may irradiate the first energy and the point beam having a designated wavelength (or type) for a designated time with a designated irradiation method, a designated delivery frequency per second (Hz), and a designated energy intensity. The designated irradiation method may include a pulse mode in which a laser is irradiated as pulses having a predetermined time interval (corresponding to the long-pulse method) and a continuous mode in which a laser is irradiated continuously (corresponding to the Q-switch method). At least one of a wavelength, an irradiation time, an irradiation method, a delivery frequency per second (Hz), and an energy intensity of the energy irradiator (210) may be set manually by a user input through the input device (240) or may be automatically set according to another setting.

The camera module (220) may include at least one image sensor of a charge coupled device (CCD) or a metal oxide semiconductor (MOS). Under control of the processor (260), the camera module (220) may capture a treatment site of a subject and generate a related image. The camera module (220) may be provided to allow adjustment of a lens direction automatically by a motor capable of controlling movement of the camera module (220), or manually by a user operation and an elastic member. Alternatively, the camera module (220) may be provided in augmented reality (AR) or virtual reality (VR) glasses or a headset worn on a forehead or above eyes of a user.

Under control of the processor (260), the display (230) may visually output at least one of images, symbols, numbers, or characters. The display (230) may include, for example, at least one of a liquid crystal display, an OLED, a touchscreen display, a hologram display, a three-dimensional display, a virtual reality display, or an augmented reality display. According to an embodiment, under control of the processor (260), the display (230) may display a processed image in which treatment regions are distinguished.

The input device (240) may detect or receive a user input of a user (e.g., a practitioner) of the electronic device (200). The input device (240) may include, for example, at least one input circuit of a push button, a tact switch, a dial switch, a touchscreen, or a microphone. The user input may be related to setting at least one of a wavelength, an irradiation time, an irradiation method, a delivery frequency per second (Hz), or an energy intensity of the first energy.

The memory (250) may store various data used by at least one component (e.g., the processor (260)) of the electronic device (200). The data may include, for example, software and input data or output data related to instructions. For example, the memory (250) may store at least one instruction for providing a laser treatment service.

According to an embodiment, the memory (250) may store region shape information related to a difference between an irradiation region of the first energy and an irradiation region of the point beam, or a difference between a skin contact region of the energy irradiator (210) and an irradiation region of the first energy. The region shape information may be provided to allow identification (e.g., calculation) of a region to which the first energy is irradiated from a point beam irradiation region or a skin contact region of the energy irradiator (210) identified from information acquired through the camera module (220). For example, the first energy may be irradiated in a circular shape having a first diameter, and the point beam may be irradiated in a circular shape having a second diameter (equal to or smaller than the first diameter) with a center overlapping a center of the irradiation region of the first energy. In this case, the region shape information may include, for example, mathematical expression information for calculating an area of a circle, region size information (e.g., the first diameter value and the second diameter value), or difference information between regions (e.g., an area ratio between a circle having the first diameter and a circle having the second diameter). The region shape information may be provided to allow identification of a treatment region to which the first energy is irradiated based on a color of the point beam included in information acquired through the camera module (220) in consideration of image distortion caused by the camera module (220). Similarly, an output end of the energy irradiator (210) may have a rectangular shape, and the first energy may be irradiated through a partial region of the rectangular output end. In this case, the region shape information may be provided to include difference information between a shape of the output end of the energy irradiator (210) and an irradiation region of the first energy.

According to an embodiment, the memory (250) may store region expression information corresponding to a spot size, a wavelength, an irradiation time, an irradiation method, and an energy intensity of the first energy. The region expression information may include color, density, and density variation information according to at least one setting of the spot size, the wavelength, the irradiation time, the irradiation method, and the energy intensity of the first energy.

The memory (250) may include various types of volatile memory or non-volatile memory. For example, the memory may include read only memory (ROM) and random access memory (RAM). In an embodiment, the memory may be located inside or outside the processor, and the memory (250) may be connected to the processor (260) through various known means.

The processor (260) may control at least one other component (e.g., a hardware or software component) of the electronic device (200) and may perform various data processing or operations. The processor (260) may include, for example, at least one of a central processing unit (CPU), a graphics processing unit (GPU), a microprocessor, an application processor, an application specific integrated circuit (ASIC), or a field programmable gate array (FPGA), and may have a plurality of cores.

According to an embodiment, the processor (260) may identify at least one setting among a wavelength, an irradiation time, an irradiation method, a delivery frequency per second (Hz), and an energy intensity of the first energy that is input by a user through the input device (240) or set as a default. According to a user input through the input device (240) (e.g., an operation of a light irradiation start button), the processor (260) may output the first energy through the energy irradiator (210) with at least one setting among a spot size, a wavelength, an irradiation time, an irradiation method, a delivery frequency per second (Hz), and an energy intensity for an irradiation time according to the at least one setting. For example, while irradiating the first energy using the energy irradiator (210), the processor (260) may continuously irradiate a point beam or irradiate the point beam for the same time as the first energy to at least a portion of the same region as an irradiation region of the first energy.

While irradiating the first energy to a treatment site of a subject, the processor (260) may cause the camera module (220) to face the treatment site and acquire information on a region to which the first energy and the point beam are irradiated or a skin contact region of the energy irradiator (210) using the camera module (220). The information acquired through the camera module (220) may include, for example, at least one of image information (e.g., a preview image or a captured image) or text information. The region to which the point beam is irradiated or the skin contact region of the energy irradiator (210) may exhibit a designated color. The designated color may correspond to a color of the point beam or an output end of the energy irradiator (210).

By analyzing the information acquired through the camera module (220), the processor (260) may identify at least one feature among a color, a shape, a pattern, movement, or other changes of the point beam or the skin contact region of the energy irradiator (210). Based on the at least one identified feature, the processor (260) may identify, from the acquired information, a treatment region corresponding to an irradiation region of the first energy within an entire treatment site.

For example, information corresponding to a region to which the point beam is irradiated or a skin contact region of the energy irradiator (210) among the information acquired through the camera module (220) may include information on at least one feature among a designated color, a shape, a pattern, movement, or other changes. Accordingly, the processor (260) may identify a region to which the point beam is irradiated or the skin contact region of the energy irradiator (210) by selecting (or identifying) the feature (e.g., the designated color, shape, pattern, movement, or other changes) from the information acquired through the camera module (220) or by recognizing the feature using artificial intelligence.

Based on region shape information stored in the memory (250), the processor (260) may identify a treatment region to which the first energy is irradiated from a region to which the point beam is irradiated. For example, when invisible first energy is irradiated in a circular spot having a diameter of 5 mm and a visible point beam is irradiated in a circular spot having a diameter of 2 mm, the processor (260) may detect an irradiation region of the point beam from the acquired information based on visual feature information of the point beam acquired through the camera module (220), and may estimate and identify an irradiation region (treatment region) of the first energy by applying a formula for calculating an area of a circle (e.g., 2π5 mm (a radius of the point beam)) based on a center of the detected region.

In an embodiment, since there may be an error generated during an image capturing process between an actual irradiation region of the first energy and an irradiation region of the point beam, the processor (260) may identify the irradiation region of the point beam from the acquired information and identify the treatment region by expanding (or reducing) the irradiation region of the point beam based on the region shape information (e.g., area difference information between a circle having a first diameter and a circle having a second diameter).

According to an embodiment, the processor (260) may display, on the display (230), a processed image corresponding to an entire treatment site such that the identified treatment region is distinguished from other regions. For example, when the treatment site is a face of a subject, the processed image may be a three-dimensional image representing the face of the subject.

According to an embodiment, the processor (260) may represent a treatment region including an entire treatment site (or at least a portion of the treatment site) of a subject with a designated color distinguished from other regions. For example, based on region expression information, the processor (260) may identify a color of the treatment region corresponding to a wavelength (or type) of the first energy and may identify a density corresponding to an irradiation amount of the first energy (an irradiation time, an irradiation method, and an energy intensity). The processor (260) may display, on the display (230), the treatment region distinguished by the identified color and density in a processed image.

According to an embodiment, the processor (260) may display color dots having a color corresponding to a wavelength (or type) of the first energy on the treatment site with a density corresponding to an irradiation amount of the first energy. For a treatment site to which energy of the first energy is repeatedly delivered, the processor (260) may add an amount of energy delivered to the site and increase a density of the color dots.

In an embodiment, the electronic device (200) may be equipment capable of performing a procedure referred to as laser toning, and the energy irradiator (210) may irradiate a laser in a circular shape and irradiate the first energy with an energy intensity and a spot size set through the input device (240).

When a practitioner (user) sets energy of 10 Hz and 1.4 J/cm2 with a spot size of 7 mm through the input device (240), the processor (260) may irradiate the first energy with an energy intensity of 10 Hz and 1.4 J/cm2 (a first energy intensity) and a spot size of 7 mm through the energy irradiator (210), and may simultaneously irradiate a point beam. While irradiating light, the processor (260) may acquire information on the treatment site using the camera module (220), and based thereon, may display a treatment region by overlapping, on a processed image (e.g., a facial image), specific color dots having a size of 7 mm with a density corresponding to the first energy intensity at a rate of ten per second (irradiation time: 10 Hz).

When the practitioner changes and sets a spot size of laser toning from 7 mm to 5 mm through the input device (240) during treatment, the processor (260) may additionally display a treatment region corresponding to a region to which the first energy is irradiated as specific color dots having a size of 5 mm. When the practitioner changes and sets a delivery frequency per second (Hz) of laser toning to 5 Hz through the input device (240), the processor (260) may additionally display a treatment site corresponding to a region to which the first energy is irradiated with a density in which five color dots are overlapped per second.

The practitioner may increase energy (energy intensity) of laser toning to 1.7 J/cm2 (a second energy intensity) through the input device (240). The processor (260) may mark specific color dots with a darker density corresponding to an increased amount of delivered energy. As described above, for a region of the treatment site to which the first energy is repeatedly irradiated to the same region, the processor (260) may increase a density of specific color dots of the region according to a degree of repetition, thereby displaying how much energy is repeatedly transmitted to the region.

In an embodiment, an output end of the energy irradiator (210) may be provided to irradiate while being in contact with skin. In this case, region shape information may be set to correspond to a shape and a size of the output end of the energy irradiator (210) in contact with the skin. The processor (260) may identify a treatment region to which the first energy is irradiated based on color and region shape information of the output end shape in contact with the skin from information acquired through the camera module (220). Based on the region shape information and the region expression information, the processor (260) may output a processed image displaying the treatment region to which the first energy is irradiated in a form of delivered energy according to a spot size, a wavelength, an irradiation time, an irradiation method, and an energy intensity using color and density.

According to an embodiment, the processor (260) may identify a position change and a state of a treatment site of a subject based on information acquired through the camera module (220). The processor (260) may display a processed image reflecting the position change and the state of the treatment site. For example, when the processor (260) identifies that a treated face is turned in a right direction based on information acquired through the camera module (220), the processor (260) may display a processed image in which a right side of the face of the subject is clearly represented.

According to various embodiments, the processor (260) may identify a point beam or a skin contact region of the energy irradiator (210) by using a software filter (or a hardware filter) capable of more clearly identifying a mark displayed on the energy irradiator (210). The software filter may operate, for example, as an algorithm capable of easily detecting the point beam or the skin contact region of the energy irradiator (210) from information acquired through the camera module (220). The hardware filter may be provided, for example, on a lens of the camera module (220) to be captured.

According to various embodiments, the processor (260) may identify a treatment region to which the first energy is irradiated by recognizing a mark displayed to overlap an output end of the point beam or the energy irradiator (210) from information acquired through the camera module (220).

For example, when the first energy is irradiated in a rectangular bar shape of 0.5 * 5 cm, and a 5 cm linear mark displayed on an output end of the energy irradiator (210) in contact with skin is captured, the processor (260) may detect a region of the mark from information acquired through the camera module (220) based on visual features of the mark, and may identify an irradiation region (treatment region) of the first energy by applying an error between the detected region and an actual irradiation region of the first energy based on region shape information.

In this case, the region shape information may be set to correspond to a shape and a size of the output end of the energy irradiator (210) in contact with the skin or a shape and a size of a designated mark. Based on a color of the output end shape or the designated mark and the region shape information, the processor (260) may identify a treatment region to which the first energy is irradiated. Based on the region shape information and the region expression information, the processor (260) may display, in a processed image, the treatment region to which the first energy is irradiated in a form of delivered energy according to a spot size, a wavelength, an irradiation time, an irradiation method, and an energy intensity using color and density.

In the above-described embodiments, the processor (260) may not generate (or store) an image of a treatment site using the camera module (220), but instead may acquire text from the camera module (220) and analyze the acquired text based on artificial intelligence. For example, the processor (260) may identify visual features of a treatment site by generating and analyzing big data related to the treatment site based on the acquired text. In this case, when generating a processed image of a treatment face of a subject, the processor (260) may generate the processed image by replacing the face of the subject with another facial shape (e.g., an avatar face). Accordingly, according to an embodiment, personal information such as a face of the subject may be protected.

According to various embodiments, the processor (260) may display a treatment region by the first energy with a color corresponding to a wavelength and an irradiation method of the first energy. The processor (260) may display a color density of each treatment region more deeply according to an irradiation amount of the first energy delivered thereto (e.g., a cumulative irradiation amount of the first energy).

According to various embodiments, among treatment regions displayed on the display (230), the processor (260) may display information on a current irradiation amount (an irradiation time, an irradiation method, and an energy intensity) of the first energy selected through the input device (240) by overlaying the information in real time on a processed image or by displaying the information as text in another region outside a face in the processed image. In addition, among the treatment regions displayed on the display (230), the processor (260) may display cumulative information on an irradiation amount (an irradiation time, an irradiation method, and an energy intensity) of the first energy selected through the input device (240) by overlaying the information on the processed image or by displaying the information as text in another region outside the face in the processed image. To this end, the processor (260) may cumulatively store, in the memory (250), information on each treatment region and an irradiation amount of the first energy for each treatment region during a specific treatment period.

According to various embodiments, when one treatment session is completed, the processor (260) may generate, automatically or according to a manual setting through the input device (240), a per-session treatment image in which treatment regions corresponding to all irradiation regions of the first energy in one treatment session are cumulatively represented. Thereafter, upon a request of a practitioner (user), the processor (260) may compare a facial image captured before treatment of a subject, a facial image captured to check progress after treatment, and the per-session treatment image. Based on a comparison result, the processor (260) may numerically display, through the display (230), a missed treatment region and a degree of a treatment effect (e.g., a pigmentation improvement rate %) in one treatment session. According to an embodiment, the electronic device (200) may numerically represent treatment results of a subject by cumulatively storing per-session treatment images over a plurality of sessions in a specific treatment period, and may thereby allow analysis of side effects in overlapped treated regions or low effects in missed treatment regions.

According to various embodiments, when the first energy is ultrasound or radio frequency energy, the energy irradiator (210) may irradiate the first energy in a state of being in contact with skin of a subject. In this case, the energy irradiator (210) may not separately irradiate a point beam, and the processor (260) may identify an output end (tip region) of the energy irradiator (210) in contact with skin of the subject through the camera module (220). Based on detecting a region at which the tip of the energy irradiator (210) contacts the skin of the subject from information acquired through the camera module (220), the processor (260) may identify (e.g., select) a treatment region. In this process, the processor (260) may distinguish and display the identified treatment region based on the region shape information and the region expression information.

According to various embodiments, the display (230) may be provided to display three-dimensionally using at least one of a virtual reality screen, an augmented reality screen, or a three-dimensional hologram. In this case, the processor (260) may capture a face of a subject in real time through the camera module (220) and implement a virtual reality screen, an augmented reality screen, or a three-dimensional hologram on the display (230). For example, the processor (260) may represent treatment regions in colors corresponding to a wavelength and an irradiation method of energy by superimposing a virtual reality screen, an augmented reality screen, or a three-dimensional hologram captured and implemented by the camera module (220) on a facial image of the subject, and may display color density to be deeper or lighter in real time according to an irradiation amount of the first energy delivered thereto (e.g., a cumulative irradiation amount of the first energy). In this regard, to assist in additionally acquiring three-dimensional coordinate information from information acquired through the camera module (220), the processor (260) may use circular or cross-shaped markers displayed in advance on an eye patch or the like. For example, the processor (260) may acquire three-dimensional coordinates for a treatment region by calculating a degree of distortion of a circular mark displayed on the eye patch or a left-right length, an up-down length, or a rotation degree of a cross-shaped mark.

A practitioner may, by using at least one of a virtual reality screen, an augmented reality screen, or a hologram, identify a color or a density of a treatment region from a three-dimensionally displayed image, thereby treating by distinguishing an irradiation region and a non-irradiation region of the first energy or distinguishing a region having insufficient irradiation amount and a region having excessive irradiation amount without turning a head to view another display screen.

According to various embodiments, the electronic device (200) may include a first device including some of components (230, 240, 250, 260) of the electronic device (200) excluding the camera module (220) and the energy irradiator (210), and the energy irradiator (210), and a second device including remaining components (230, 240, 250, 260) of the electronic device (200) excluding the energy irradiator (210), and the camera module (220).

According to various embodiments, the processor (260) may acquire setting values of the energy irradiator (210) (e.g., at least one of a type of the first energy, a medium, a spot size, a wavelength, a pulse width, or an energy intensity) displayed on a display of the first device based on an image captured using the camera module (220). Based on the setting values of the energy irradiator (210), the processor (260) may identify a treatment region corresponding to an irradiation region of the first energy, and may display, on the display, a processed image corresponding to a treatment site of the subject such that the treatment region is distinguished from other regions.

According to various embodiments, when the first device and the second device are connected through a designated communication channel, the first device and the second device may transmit and receive data related to setting values of the first energy to and from each other through the designated communication channel.

As described above, the electronic device (200) according to an embodiment may, when using the first energy that does not significantly cause visually observable symptoms (e.g., redness) immediately after being irradiated onto a treatment site, support a practitioner to intuitively identify the treatment site and energy delivered thereto based on detection of a point beam or an output end exhibiting a specific color, shape, pattern, movement, or other changes.

In addition, when the energy irradiator (210) does not irradiate a point beam, the electronic device (200) according to an embodiment may additionally irradiate a point beam to a first energy delivery region or identify a treatment site to which the first energy is delivered based on a contact surface with skin, and may visually display the treatment site, thereby supporting accurate treatment by the practitioner.

Furthermore, the electronic device (200) according to an embodiment may cumulatively image treatment regions corresponding to treatment results of a subject (e.g., per-session treatment images) and may also quantify treatment effects, thereby supporting the practitioner in finding a treatment method that is more necessary, suitable, and efficient for the subject.

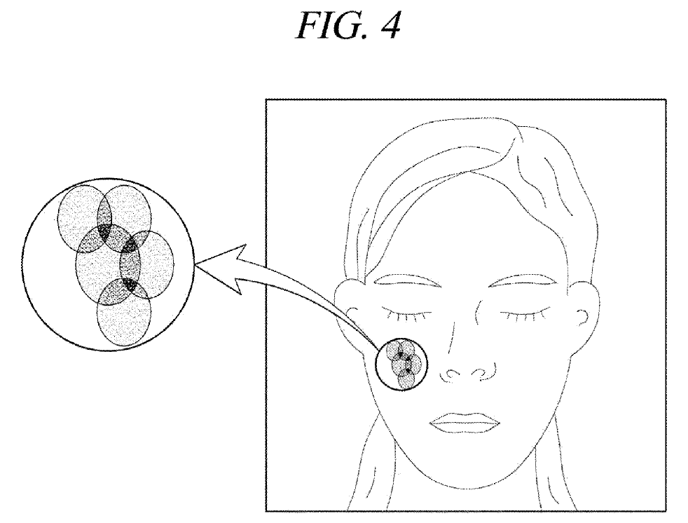

FIG. 4 illustrates an example of displaying an irradiation region of the first energy according to an embodiment.

Referring to FIG. 4, the energy irradiator (210) may irradiate energy having a first intensity and a second intensity in circular shapes having a first size and a second size according to a setting through the input device (240). In this case, the electronic device (200) may display treatment regions on a face of a subject (patient) in circular shapes having the first size and the second size. In addition, treatment regions to which a larger amount of the first energy is delivered may be displayed with a darker density.

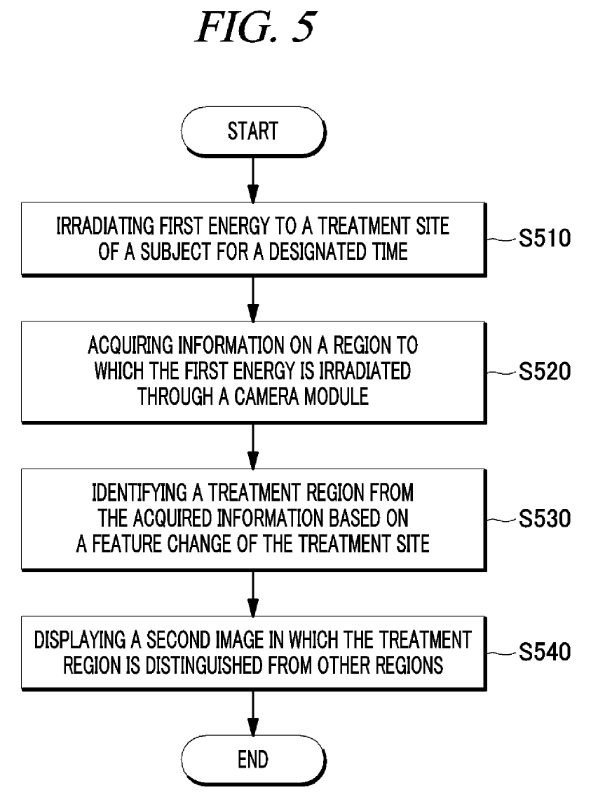

FIG. 5 is a flowchart illustrating a method for displaying a treatment site according to an embodiment.

Referring to FIG. 5, in operation 510, the electronic device (200) may irradiate the first energy to a portion of an entire treatment site of a subject for a predetermined time. The first energy may provide a designated therapeutic effect to treated skin. The first energy may be a laser having a designated wavelength.

In operation 520, the electronic device (200) may cause the camera module (220) to face the treatment site and acquire information on a region to which the first energy is irradiated through the camera module (220). For example, while irradiating the first energy, the electronic device (200) may irradiate a point beam to the same region as the first energy and acquire information on a position by capturing a region to which the point beam is irradiated. The point beam may be irradiated to at least a portion of the same region as the first energy so as to exhibit a designated color, a shape (e.g., circular), a pattern (e.g., a grid pattern or blinking), movement, or other changes. In this case, information acquired through the camera module (220) may include, for example, information on visual features corresponding to a region to which the point beam is irradiated, such as a designated color according to a wavelength of the point beam, a shape, a pattern, movement, or other changes.

In operation 530, the electronic device (200) may identify, among the entire treatment site, a region to which the first energy is irradiated based on features of information acquired through the camera module (220). For example, the electronic device (200) may identify a region to which a point beam is irradiated from the acquired information based on at least one visual feature among a color, a shape, a pattern, movement, or other changes of the point beam, and may identify a region to which the first energy is irradiated, that is, a treatment region, based on a difference between irradiation regions of the first energy and the point beam according to region shape information. As another example, when the energy irradiator (210) does not irradiate a point beam, the electronic device (200) may additionally irradiate a point beam to a first energy delivery region or may identify a region to which the first energy is irradiated based on a skin contact surface (or an output end) of the energy irradiator (210) with respect to the treatment site of the subject.

In operation 540, the electronic device (200) may display a processed image corresponding to at least a currently treated region of the subject such that the identified treatment region is distinguished from other regions. For example, the electronic device (200) may distinguish and display the treatment region by adjusting a color and a density of the treatment region based on a spot size, a wavelength, an irradiation time, an irradiation method, and an energy intensity of the first energy.

As described above, when using the first energy that does not significantly cause visually observable symptoms (e.g., redness) immediately after being irradiated onto a treatment site, the electronic device (200) according to an embodiment may display a processed image that allows intuitive identification of the treatment site and energy delivered thereto based on detection of a point beam having a color tone or detection of an output end.

Various embodiments of the present document and terms used therein are not intended to limit technical features described in the present document to specific embodiments, and should be understood to include various modifications, equivalents, or alternatives of the embodiments. In relation to the description of the drawings, similar or related components may be denoted by similar reference numerals. A singular form of a noun corresponding to an item includes one or more items unless the context clearly indicates otherwise. In the present document, phrases such as “A or B”, “at least one of A and B”, “at least one of A or B”, “A, B, or C”, “at least one of A, B, and C”, and “at least one of A, B, or C” each include any one of the listed items or all possible combinations thereof. Terms such as “first”, “second”, “firstly”, or “secondly” are used merely to distinguish one component from another and do not limit the components in other aspects (e.g., importance or order). When a certain (e.g., first) component is referred to as being “coupled” or “connected” to another (e.g., second) component, with or without the terms “functionally” or “communicatively”, it means that the certain component may be directly connected (e.g., wired), wirelessly connected, or connected through a third component to the other component.

In the present document, the term “module” may include a unit implemented in hardware, software, or firmware, and may be interchangeably used with terms such as logic, a logic block, a component, or a circuit. A module may be an integrally formed component or a minimum unit of the component, or a part thereof, that performs one or more functions. For example, according to an embodiment, a module may be implemented in a form of an application-specific integrated circuit (ASIC).

Various embodiments of the present document may be implemented as software (e.g., a program) including one or more instructions stored in a machine-readable storage medium (e.g., the memory 250 of FIG. 3) (e.g., an internal memory or an external memory) readable by a machine (e.g., an electronic device). For example, a processor (e.g., the processor (260)) of a machine (e.g., the electronic device (200)) may invoke and execute at least one instruction among one or more instructions stored in the storage medium. This allows the machine to be operated to perform at least one function according to the invoked at least one instruction. The one or more instructions may include code generated by a compiler or code executable by an interpreter. A machine-readable storage medium may be provided in a form of a non-transitory storage medium. Here, “non-transitory” means only that the storage medium is a tangible device and does not include a signal (e.g., an electromagnetic wave), and the term does not distinguish between a case in which data is stored in the storage medium semi-permanently and a case in which data is stored temporarily.

According to an embodiment, a method according to various embodiments disclosed in the present document may be provided by being included in a computer program product. The computer program product may be traded as a commodity between a seller and a purchaser. The computer program product may be distributed in a form of a machine-readable storage medium (e.g., a compact disc read only memory (CD-ROM)), or may be distributed online (e.g., downloaded or uploaded) through an application store (e.g., Play Store™) or directly between two user devices (e.g., smartphones). In the case of online distribution, at least a portion of the computer program product may be at least temporarily stored or temporarily generated in a machine-readable storage medium such as a memory of a manufacturer’s server, a server of an application store, or a relay server.

Components according to various embodiments of the present document may be implemented in software or in hardware such as a digital signal processor (DSP), a field programmable gate array (FPGA), or an application specific integrated circuit (ASIC), and may perform predetermined roles. The “components” are not limited to software or hardware, and each component may be configured to reside in an addressable storage medium or may be configured to reproduce one or more processors. As an example, components may include software components, object-oriented software components, class components, and task components, and processes, functions, attributes, procedures, subroutines, segments of program code, drivers, firmware, microcode, circuits, data, databases, data structures, tables, arrays, and variables.

According to various embodiments, each of the above-described components (e.g., modules or programs) may include a single entity or a plurality of entities. According to various embodiments, one or more of the above-described components or operations may be omitted, or one or more other components or operations may be added. Alternatively or additionally, a plurality of components (e.g., modules or programs) may be integrated into one component. In this case, the integrated component may perform one or more functions of each of the plurality of components in the same or similar manner as those performed by the respective components prior to integration. According to various embodiments, operations performed by a module, a program, or another component may be executed sequentially, in parallel, repeatedly, or heuristically, or one or more of the operations may be executed in a different order, omitted, or one or more other operations may be added.

What is claimed is:

1. An electronic device comprising:
a display;
a camera module; and
a processor functionally connected to the display and the camera module,
wherein the processor is configured to identify a treatment region corresponding to an irradiation region to which a first energy determined by an energy irradiator is irradiated, and to display, on the display, a processed image corresponding to a treatment site of a subject such that the treatment region is distinguished from other regions.

2. The electronic device of claim 1,
wherein the processor is configured to display, in the processed image, the treatment region in a color distinguished from the other regions, and to adjust and display a density of the color according to an irradiation amount of the first energy.

3. The electronic device of claim 1, further comprising:
the energy irradiator; and
an input device configured to adjust at least one setting among a spot size, a wavelength, an irradiation time, an irradiation method, a delivery frequency per second (Hz), and an output energy intensity of the first energy according to a designated therapeutic effect,
wherein the processor is configured to irradiate, through the energy irradiator, the first energy corresponding to the at least one setting input through the input device.

4. The electronic device of claim 1, wherein the processor is configured to:
identify at least one setting related to a type of the first energy and an amount of the first energy delivered;
display the treatment region in a color corresponding to the type of the first energy; and
display the treatment region in the processed image with a density corresponding to the amount of the first energy delivered according to the at least one setting.

5. The electronic device of claim 1,
wherein the processor is configured to identify a visual feature change of the treatment site based on visual features of a point beam irradiated to a same region as the first energy.

6. The electronic device of claim 5, wherein the processor is configured to:
obtain pre-stored region shape information, the region shape information being related to a difference between irradiation regions of the first energy and the point beam and to image distortion caused by the camera module; and
identify an irradiation region of the point beam included in the obtained information based on the region shape information, and identify the treatment region to which the first energy is irradiated based on the identified irradiation region.

7. The electronic device of claim 1, wherein the processor is configured to:
obtain, using the camera module, setting values of the energy irradiator including at least one of a type, a wavelength, an irradiation time, an irradiation method, and an output energy intensity of the first energy; and
display, in the processed image, the treatment region distinguished from other regions based on the obtained setting values.

8. The electronic device of claim 1, wherein the processor is configured to:
obtain, using the camera module, information on an output end of the energy irradiator in contact with the treatment site of the subject;
identify a skin contact region of the energy irradiator from the obtained information; and
identify the treatment region based on region shape information related to a difference between the skin contact region and an irradiation region of the first energy.

9. The electronic device of claim 1,
wherein the processed image is a two-dimensional or three-dimensional image of a face of the subject.

10. The electronic device of claim 1,
wherein the processor is configured to store and record a position change and a cumulative state of the treatment site based on the obtained information, and to display the processed image reflecting the position change and the cumulative state.
